(19) United States
(12) Patent Application Publication
(10) Pub. No.: US 2019/0142767 A1
(43) Pub. Date: May 16, 2019

(54) ABUSE-PROOFED ORAL DOSAGE FORM

(71) Applicant: GRUNENTHAL GMBH, Aachen (DE)

(72) Inventors: Johannes BARTHOLOMÄUS, Aachen (DE); Heinrich KUGELMANN, Aachen (DE); Elisabeth ARKENAU-MARIC, Köln (DE)

(73) Assignee: GRUNENTHAL GMBH, Aachen (DE)

(21) Appl. No.: 16/243,205

(22) Filed: Jan. 9, 2019

Related U.S. Application Data
(63) Continuation of application No. 15/956,867, filed on Apr. 19, 2018, now abandoned.
Application No. 15/956,867 is a continuation of application No. 15/095,336, filed on Apr. 11, 2016, now abandoned.
Application No. 15/095,336 is a continuation of application No. 14/874,931, filed on Oct. 5, 2015, now abandoned.
Application No. 14/874,931 is a continuation of application No. 14/685,718, filed on Apr. 14, 2015, now abandoned.
Application No. 14/685,718 is a continuation of application No. 14/508,262, filed on Oct. 7, 2014, now abandoned.
Application No. 14/508,262 is a continuation of application No. 14/168,159, filed on Jan. 30, 2014, now abandoned.
Application No. 14/168,159 is a continuation of application No. 13/927,266, filed on Jun. 26, 2013, now abandoned.
Application No. 13/927,266 is a continuation of application No. 10/890,707, filed on Jul. 14, 2004, now abandoned.

(30) Foreign Application Priority Data
Jul. 1, 2004 (DE) 10 2004 032 103.5

(51) Int. Cl. A61K 31/137; A61K 9/28; A61K 9/00; A61K 9/48; A61K 9/20; A61K 47/38; A61K 31/135; A61K 47/10

(52) U.S. Cl. CPC A61K 31/137; A61K 9/2893; A61K 9/0053; A61K 9/4808; A61K 9/2077; A61K 47/38; A61K 47/10; A61K 9/20; A61K 9/2095; A61K 31/135; A61K 9/2054; A61K 9/2031; A61K 9/2013

(57) ABSTRACT

The present invention relates to an abuse-proofed oral dosage form with controlled release of (1R,2R)-3-(3-dimethylamino-1-ethyl-2-methyl-propyl)phenol for once daily administration, which comprises the active ingredient and/or one or more of the pharmaceutically acceptable salts thereof (A), at least one synthetic or natural polymer (C), delayed-release auxiliary substances, optionally physiologically acceptable auxiliary substances (B) and optionally a wax (D), component (C) or (D) in each case exhibiting a breaking strength of at least 500 N, preferably of at least 1000 N.

This application is a continuation of U.S. patent application Ser. No. 15/956,867, filed Apr. 19, 2018, now pending, which is a continuation of U.S. patent application Ser. No. 15/095,336, filed on Apr. 11, 2016, now abandoned, which is a continuation of U.S. patent application Ser. No. 14/874,931, filed on Oct. 5, 2015, now abandoned, which is a continuation of U.S. patent application Ser. No. 14/685,718 filed Apr. 14, 2015, now abandoned, which is a continuation of U.S. patent application Ser. No 14/508,262, filed Oct. 7, 2014, now abandoned, which is a continuation of U.S. patent application Ser. No. 14/168,159, filed Jan. 30, 2014, now abandoned, which is a continuation of U.S. patent application Ser. No. 13/927,266, filed Jun. 26, 2013, now abandoned, which is a continuation of U.S. patent application Ser. No. 10/890,707, filed Jul. 14, 2004, now abandoned, which, in turn, claims priority of German Patent Application No. 10 2004 032 103.5, filed Jul. 1, 2004.

The present invention relates to an abuse-proofed oral dosage form with controlled release of the active ingredient (1R,2R)-3-(3-dimethylamino-1-ethyl-2-methyl-propyl)phenol for once daily administration, which dosage form comprises the active ingredient and/or one or more of the pharmaceutically acceptable salts thereof (A), at least one synthetic or natural polymer (C), delayed-release auxiliary substances, optionally physiologically acceptable auxiliary substances (B) and optionally a wax (D), component (C) or (D) in each case exhibiting a breaking strength of at least 500 N, preferably of 1000 N.

This active ingredient also exhibits, apart from an excellent pain-relieving action, abuse potential, i.e. it may be used by an abuser to bring about an action which does not correspond to its intended purpose. This active ingredient is accordingly used by abusers, for example, to induce a state of narcosis or euphoria.

These dosage forms containing active ingredient are frequently used for long-term treatment, for example for chronic pain or pain caused by tumours. In long-term treatment, in particular, it is important to enable the patient to enjoy a good quality of life. The measures which improve the quality of life of a patient include dosage forms which allow once daily administration. However, because of the relatively large quantity of active ingredient, such dosage forms, which provide delayed release of the active ingredient, are particularly attractive to the abuser in order to induce the desired state of narcosis or euphoria as quickly as possible.

Since, however, delayed-release dosage forms containing the stated active ingredient do not usually give rise to the kick desired by the abuser when taken orally even in abusively high quantities, these dosage forms for example in the form of tablets or capsules are also comminuted, e.g. ground, and sniffed by the abuser for the purpose of abuse or the active ingredients are preferably extracted from the powder obtained in this way by means of an aqueous liquid and the resultant solution is administered parenterally, in particular intravenously, optionally after filtration through cotton wool or cellulose wadding. This type of administration produces further accelerated increase in active ingredient level than with oral or nasal abuse, with the result desired by the abuser, namely the kick.

US-A-4,070,494 proposed adding a swellable agent to the dosage form in order to prevent abuse. When water is added to extract the active ingredient used therein, this agent swells and ensures that the filtrate separated from the gel contains only a small quantity of active ingredient.

The multilayer tablet disclosed in WO 95/20947 is based on a similar approach to preventing parenteral abuse, said tablet containing an active ingredient with potential for abuse and at least one gel former, each in different layers.

WO 03/015531 A2 discloses another approach to preventing parenteral abuse. A dosage form containing an analgesic opioid and a dye as an aversive agent is described therein. The colour released by tampering with the dosage form is intended to discourage the abuser from using the dosage form which has been tampered with.

Another known option for complicating abuse involves adding to the dosage form an antagonist to the active ingredient, such as for example naloxone or naltexone, or compounds which cause a physiological defence response, such as for example ipecacuanha (ipecac) root, or bitter substances.

Since, however, as in the past, it is in most cases necessary for the purposes of abuse to pulverise dosage forms with controlled release of the active ingredient, it was the object of the present invention to complicate or prevent the pulverisation which precedes abuse of dosage forms with controlled release of (1R,2R)-3-(3-dimethylamino-1-ethyl-2-methyl-propyl)phenol with the means conventionally available for potential abuse and in this manner to provide a dosage form for the active ingredient, which, when correctly administered, ensures the desired therapeutic action with once daily administration, but from which the active ingredient cannot be converted into a form suitable for abuse simply by pulverisation.

This object has been achieved by the provision of the abuse-proofed, oral dosage form according to the invention with controlled release of (1R,2R)-3-(3-dimethylamino-1-ethyl-2-methyl-propyl)phenol for once daily administration, which dosage form, apart from the active ingredient and/or one or more of the pharmaceutically acceptable compounds thereof, preferably salts or derivatives, preferably esters or ethers and the corresponding stereoisomers and/or pharmaceutically acceptable compounds or derivatives (A), comprises at least one synthetic or natural polymer (C), delayed-release auxiliary substances (E), optionally physiologically acceptable auxiliary substances (B) and optionally a wax (D), component (C) or (D) in each case exhibiting a breaking strength of at least 500 N, preferably of 1000 N.

By using components (C) and optionally (D) with the stated minimum breaking strength, preferably in such quantities that the dosage form also exhibits such a minimum breaking strength, pulverisation of the dosage form with conventional means and thus subsequent abuse, preferably nasal or parenteral abuse, may be considerably complicated or prevented.

Preferably, the components (C) and optionally (D) are present in such quantities that the dosage form exhibits a breaking strength of at least 500 N, preferably at least 1000 N.

Without sufficient comminution of the dosage form, non-hazardous parenteral, in particular intravenous or nasal administration is impossible or extraction of the active ingredient takes the abuser too long, or no or an inadequate kick is obtained on abusive oral administration, since spontaneous release does not occur.

According to the invention, comminution is taken to mean pulverisation of the dosage form with conventional means which are conventionally available to an abuser, such as for example a pestle and mortar, a hammer, a mallet or other usual means for pulverisation by application of force.

The dosage form according to the invention is thus suitable for preventing the parenteral, nasal and/or oral abuse of (1R,2R)-3-(3-dimethylamino-1-ethyl-2-methyl-propyl)phenol. The active ingredient is known from EP-A-0 693 475 as an analgesically active pharmaceutical preparation.

The active ingredient (1R,2R)-3-(3-dimethylamino-1-ethyl-2-methyl-propyl)phenol may not only be used as such, i.e. as the free base, but also in the form of a pharmaceutically acceptable salt, for example as the hydrochloride and as a corresponding derivative, in particular as an amide, ester or ether. Production of the active ingredient is also known from EP-A-0 693 475 A1.

In the dosage form according to the invention, the content of active ingredient is preferably between 0.5 and 80 wt. %, particularly preferably between 10 and 40 wt. % and very particularly preferably between 5-50 wt. %.

The dosage form according to the invention contains (1R,2R)-3-(3-dimethylamino-1-ethyl-2-methyl-propyl)phenol as such and/or as a pharmaceutically acceptable salt conventionally in a quantity of 2.5 to 1,000 mg, in particular of 5 to 800 mg, very particularly preferably of 5-600 mg calculated as (1R,2R)-3-(3-dimethylamino-1-ethyl-2-methyl-propyl)phenol per dosage form or dosage unit.

According to the invention, pharmaceutically acceptable salts of the active ingredient are salts which, when used pharmaceutically, in particular when correctly administered to mammals or humans, in particular humans, are physiologically acceptable. Such pharmaceutical salts may, for example, be formed with inorganic or organic acids. A hydrochloride is preferably used as the salt.

In order to achieve the necessary breaking strength of the dosage form according to the invention, at least one synthetic, semi-synthetic or natural polymer (C) is used which has a breaking strength, measured using the method disclosed in the present application, of at least 500 N, preferably of 1000 N. Preferably, at least one polymer is selected for this purpose from among the group comprising polyalkylene oxides, preferably polymethylene oxides, polyethylene oxides, polypropylene oxides, polyolefins, preferably polyethylenes, polypropylenes, polyvinyl chlorides, polycarbonates, polystyrenes, polymethacrylates, the copolymers thereof, and mixtures of at least two of the stated polymer classes or polymers. Particularly preferably, a water-soluble or water-swellable polymer is used. The polymers are distinguished by a molecular weight of at least 0.5 million, preferably of at least 1 million, particularly preferably of up to 15 million, determined by rheological measurement. Particularly preferably suitable are thermoplastic polyalkylene oxides, such as polyethylene oxide, with a molecular weight of at least 0.5 million, preferably of at least 1 million, particularly preferably of up to 15 million, determined by rheological measurement. The polyethylene oxides have a viscosity at 25° C. of 4500 to 17600 cP, measured on a 5 wt. % aqueous solution of the polymer using a model RVF Brookfield viscosimeter (spindle no. 2/rotational speed 2 rpm), of 400 to 4000 cP, measured on a 2 wt. % aqueous solution of the polymer using the stated viscosimeter (but with spindle no. 1 or 3/rotational speed 10 rpm) or of 1650 to 10000 cP, measured on a 1 wt. % aqueous solution of the polymer using the stated viscosimeter (but with spindle no. 2/rotational speed 2 rpm).

The polymers are preferably used as powder to produce the dosage form according to the invention.

Moreover, in addition to the above-stated polymers, at least one natural, semi-synthetic or synthetic wax (D) with a breaking strength, measured using the method disclosed in the present application, of at least 500 N, preferably of 1000 N, may additionally be used to achieve the necessary breaking strength of the dosage form according to the invention. Waxes with a softening point of at least 60° C. are preferred. Carnauba wax and beeswax are particularly preferred. Carnauba wax is very particularly preferred. Carnauba wax is a natural wax which is obtained from the leaves of the carnauba palm and has a softening point of at most 90° C. When additionally using the wax component, the latter is used together with at least one polymer (C), preferably a polyethylene oxide, in such quantities that the dosage form exhibits a breaking strength of at least 500 N, preferably of 1000 N, measured using the method stated in the present application.

The dosage forms according to the invention are distinguished in that they cannot be pulverised using conventional comminution tools, such as grinders, due to their hardness. Oral, parenteral, in particular intravenous, or nasal abuse is complicated a very great deal thereby, if not ruled out altogether. However, in order to prevent any possible abuse of the dosage forms according to the invention, in a preferred embodiment, the dosage forms according to the invention may contain further abuse-complicating or -preventing agents as auxiliary substances (B).

Thus, the abuse-proofed dosage form according to the invention may comprise, in addition to the active ingredient used according to the invention, at least one polymer (C) and optionally at least one wax (D), at least one of the following components (a)-(e) as auxiliary substances (B):
(a) at least one substance which irritates the nasal passages and/or pharynx,
(b) at least one viscosity-increasing agent, which, with the assistance of a necessary minimum quantity of an aqueous liquid, preferably as an aqueous extract obtained from the dosage form, forms a gel which preferably remains visually distinguishable when introduced into a further quantity of an aqueous liquid,
(c) at least one opioid antagonist for the active ingredient used,
(d) at least one emetic,
(e) at least one dye as an aversive agent,
(f) at least one bitter substance.

The components (a) to (f) are each additionally suitable on their own as additional protection of the dosage form according to the invention against abuse. Accordingly, component (a) is preferably suitable for proofing the dosage form against nasal, oral and/or parenteral, preferably intravenous, abuse, component (b) is preferably suitable for proofing against parenteral, particularly preferably intravenous and/or nasal abuse, component (c) is preferably suitable for proofing against nasal and/or parenteral, particularly preferably intravenous, abuse, component (d) is preferably suitable for proofing against parenteral, particularly preferably intravenous, and/or oral and/or nasal abuse, component (e) is suitable as a visual deterrent against oral or parenteral abuse and component (f) is suitable for proofing against oral or nasal abuse. Through the co-use of at least one of the above-stated components, it is possible to complicate abuse even more effectively for the dosage forms according to the invention.

In one embodiment, the dosage form according to the invention may also comprise two or more of components (a)-(f) in a combination, preferably in the combinations (a), (b) and optionally (c) and/or (f) and/or (e) or (a), (b) and optionally (d) and/or (f) and/or (e).

In another embodiment, the dosage form according to the invention may comprise all of components (a)-(f).

If the dosage form according to the invention comprises component (a) as additional protection against abuse, substances which irritate the nasal passages and/or pharynx which may be considered according to the invention are any substances which, when administered via the nasal passages and/or pharynx, bring about a physical reaction which is either so unpleasant for the abuser that he/she does not wish to or cannot continue administration, for example burning, or physiologically counteracts taking of the active ingredient, for example due to increased nasal secretion or sneezing. These substances which conventionally irritate the nasal passages and/or pharynx may also bring about a very unpleasant sensation or even unbearable pain when administered parenterally, in particular intravenously, such that the abuser does not wish to or cannot continue taking the substance.

Particularly suitable substances which irritate the nasal passages and/or pharynx are those which cause burning, itching, an urge to sneeze, increased formation of secretions or a combination of at least two of these stimuli. Appropriate substances and the quantities thereof which are conventionally to be used are known per se to the person skilled in the art or may be identified by simple preliminary testing.

The substance which irritates the nasal passages and/or pharynx of component (a) is preferably based on one or more constituents or one or more plant parts of at least one hot substance drug.

Corresponding hot substance drugs are known per se to the person skilled in the art and are described, for example, in “Pharmazeutische Biologie—Drogen and ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

One or more constituents of at least one hot substance drug selected from the group consisting of Allii sativi bulbus (garlic), Asari rhizoma cum herba (Asarum root and leaves), Calami rhizoma (calamus root), Capsici fructus (capsicum), Capsici fructus acer (cayenne pepper), Curcumae longae rhizoma (turmeric root), Curcumae xanthorrhizae rhizoma (Javanese turmeric root), Galangae rhizoma (galangal root), Myristicae semen (nutmeg), Piperis nigri fructus (pepper), Sinapis albae semen (white mustard seed), Sinapis nigri semen (black mustard seed), Zedoariae rhizoma (zedoary root) and Zingiberis rhizoma (ginger root), particularly preferably from the group consisting of Capsici fructus (capsicum), Capsici fructus acer (cayenne pepper) and Piperis nigri fructus (pepper) may preferably be added as component (a) to the dosage form according to the invention.

The constituents of the hot substance drugs preferably comprise o-methoxy(methyl)phenol compounds, acid amide compounds, mustard oils or sulfide compounds or compounds derived therefrom.

Particularly preferably, at least one constituent of the hot substance drugs is selected from the group consisting of myristicin, elemicin, isoeugenol, α-asarone, safrole, gingerols, xanthorrhizol, capsaicinoids, preferably capsaicin, capsaicin derivatives, such as N-vanillyl-9E-octadecenamide, dihydrocapsaicin, nordihydrocapsaicin, homocapsaicin, norcapsaicin and nomorcapsaicin, piperine, preferably trans-piperine, glucosinolates, preferably based on non-volatile mustard oils, particularly preferably based on p-hydroxybenzyl mustard oil, methylmercapto mustard oil or methylsulfonyl mustard oil, and compounds derived from these constituents.

The dosage form according to the invention may preferably also contain plant parts of the corresponding hot substance drugs in a quantity of 0.01 to 30 wt. %, particularly preferably of 0.1 to 0.5 wt. %, in each case relative to the total weight of the dosage unit.

If one or more constituents of corresponding hot substance drugs are used, the quantity thereof in a dosage unit according to the invention preferably amounts to 0.001 to 0.005 wt. %, relative to the total weight of the dosage unit. A dosage unit is taken to mean a separate or separable administration unit, such as for example a tablet or a capsule.

Another option for preventing abuse of the dosage form according to the invention consists in adding at least one viscosity-increasing agent as a further abuse-preventing component (b) to the dosage form, which, with the assistance of a necessary minimum quantity of an aqueous liquid, preferably as an aqueous extract obtained from the dosage form, forms a gel which is virtually impossible to administer safely and preferably remains visually distinguishable when introduced into a further quantity of an aqueous liquid.

For the purposes of the present application visually distinguishable means that the active ingredient-containing gel formed with the assistance of a necessary minimum quantity of aqueous liquid, when introduced, preferably with the assistance of a hypodermic needle, into a further quantity of aqueous liquid at 37° C., remains substantially insoluble and cohesive and cannot straightforwardly be dispersed in such a manner that it can safely be administered parenterally, in particular intravenously. The material preferably remains visually distinguishable for at least one minute, preferably for at least 10 minutes.

Increasing the viscosity to a gel makes it more difficult or even impossible for it to be passed through a needle or injected. If the gel remains visually distinguishable, this means that the gel obtained on introduction into a further quantity of aqueous liquid, for example by injection into blood, initially remains in the form of a largely cohesive thread, which, while it may indeed be broken up mechanically into smaller fragments, cannot be dispersed or even dissolved in such a manner that it can safely be administered parenterally, in particular intravenously. In combination with at least one further present component (a), (d) to (f), this additionally leads to unpleasant burning, vomiting, bad flavour and/or visual deterrence.

Intravenous administration of such a gel would most probably result in obstruction of blood vessels, associated with serious damage to the health of the abuser.

In order to verify whether a viscosity-increasing agent is suitable as component (b) for use in the dosage form according to the invention, the active ingredient is mixed with the viscosity-increasing agent and suspended in 10 ml of water at a temperature of 25° C. If this results in the formation of a gel which fulfils the above-stated conditions, the corresponding viscosity-increasing agent is suitable for additionally preventing or averting abuse of the dosage forms according to the invention.

If component (b) is added to the dosage form obtained by the process according to the invention, preferably one or more viscosity-increasing agents are used, which are selected from the group comprising microcrystalline cellulose with 11 wt. % carboxymethylcellulose sodium (Avicel® RC 591), carboxymethylcellulose sodium (Blanose®, CMC-Na C300P®, Frimulsion BLC-5®, Tylose C300 P®), polyacrylic acid (Carbopol® 980 NF, Carbopol® 981), locust bean flour (Cesagum® LA-200, Cesagum® LID/150, Cesagum® LN-1), pectins, preferably from citrus fruits or apples(Cesapectin® HM Medium Rapid Set), waxy maize starch (C*Gel)04201®), sodium alginate (Frimulsion ALG (E401)®) guar flour (Frimulsion BM®, Polygum 26/1-75®), iota-carrageenan (Frimulsion D021®), karaya gum, gellan gum (Kelcogel F®), Kelcogel LT100®, galactomannan (Meyprogat 150®), tara stone flour (Polygum 43/1®), propylene glycol alginate (Protanal-Ester SD-LB®), sodium-hyaluronate, tragacanth, tara gum (Vidogum SP 200®), fermented polysaccharide welan gum (K1A96), xanthans such as xanthan gum (Xantural 180®). Xanthans are particularly preferred. The names stated in brackets are the trade names by which the materials are known commercially. In general, a quantity of 0.1 to 5 wt. %, relative to the total quantity of the dosage form, of the stated viscosity-increasing agent(s) is sufficient to fulfil the above-stated conditions.

The component (b) viscosity-increasing agents, where provided, are preferably present in the dosage form according to the invention in quantities of mg per dosage unit, i.e. per administration unit.

In a particularly preferred embodiment of the present invention, the viscosity-increasing agents used as component (b) are those which, preferably by extraction from the dosage form with the necessary minimum quantity of aqueous liquid, form a gel which encloses air bubbles. The resultant gels are distinguished by a turbid appearance, which provides the potential abuser with an additional optical warning and discourages him/her from administering the gel parenterally.

Component (C) may also optionally serve as an additional viscosity-increasing agent, which forms a gel with the assistance of a necessary minimum quantity of aqueous liquid.

It is also possible, to arrange the viscosity-increasing component and the other constituents of the dosage form according to the invention spatially separately from one another.

Moreover, in order to discourage and prevent abuse, the dosage form according to the invention may furthermore comprise component (c), namely one or more antagonists for the active ingredient used, wherein the antagonist is preferably spatially separated from the remaining constituents of the dosage form according to the invention and, when correctly used, should not exert any effect.

Suitable antagonists for preventing the abuse of the active ingredient used are known per se to the person skilled in the art and may be present in the dosage form according to the invention as such or in the form of corresponding derivatives, in particular esters or ethers, or in each case in the form of corresponding physiologically acceptable compounds, in particular in the form of the salts or solvates thereof.

The antagonist used is preferably selected from the group comprising naloxone, naltrexone, nalmefene, nalide and nalmexone, in each case optionally in the form of a corresponding physiologically acceptable compound, in particular in the form of a base, a salt or solvate. The corresponding antagonists, where component (c) is provided, are preferably used in a quantity of ≥1 mg, particularly preferably in a quantity of 3 to 100 mg, very particularly preferably in a quantity of 5 to 50 mg per dosage form, i.e. per administration unit.

The dosage form according to the invention preferably comprises the antagonist component in a conventional therapeutic dose known to the person skilled in the art, particularly preferably in a quantity of twice to three times this dose per administration unit.

If the combination for additional discouragement and prevention of abuse of the dosage form according to the invention comprises component (d), it may comprise at least one emetic, which is preferably present in a spatially separated arrangement from the other components of the dosage form according to the invention and, when correctly used, is intended not to exert its effect in the body.

Suitable emetics for additionally preventing abuse of the dosage form according to the invention are known per se to the person skilled in the art and may be present in the dosage form according to the invention as such or in the form of corresponding derivatives, in particular esters or ethers, or in each case in the form of corresponding physiologically acceptable compounds, in particular in the form of the salts or solvates thereof.

An emetic based on one or more constituents of ipecacuanha (ipecac) root, preferably based on the constituent emetine, may preferably be considered for the dosage form according to the invention, as are, for example, described in “Pharmazeutische Biologie—Drogen und ihre Inhaltsstoffe” by Prof. Dr. Hildebert Wagner, 2nd, revised edition, Gustav Fischer Verlag, Stuttgart, New York 1982. The corresponding literature description is hereby introduced as a reference and is deemed to be part of the disclosure.

The dosage form according to the invention may preferably comprise the emetic emetine as component (d), preferably in a quantity of 3 mg, particularly preferably of 10 mg and very particularly preferably in a quantity of 20 mg per dosage form, i.e. administration unit.

Apomorphine may likewise preferably be used as an emetic for additional abuse-proofing, preferably in a quantity of preferably 3 mg, particularly preferably of 5 mg and very particularly preferably of 7 mg per administration unit.

If the dosage form according to the invention contains component (e) as an additional abuse-preventing auxiliary substance, the use of such a dye brings about an intense coloration of a corresponding aqueous solution, in particular when the attempt is made to extract the active ingredient for parenteral, preferably intravenous administration, which coloration may act as a deterrent to the potential abuser. Oral abuse, which conventionally begins by means of aqueous extraction of the active ingredient, may also be prevented by this coloration. Suitable dyes and the quantities required for the necessary deterrence may be found in WO 03/015531, wherein the corresponding disclosure should be deemed to be part of the present disclosure and is hereby introduced as a reference.

If the dosage form according to the invention contains component (f) as a further abuse-preventing auxiliary substance, this addition of at least one bitter substance and the consequent impairment of the flavour of the dosage form additionally prevents oral and/or nasal abuse.

Suitable bitter substances and the quantities effective for use may be found in US-2003/0064099, the corresponding disclosure of which should be deemed to be the disclosure of the present application and is hereby introduced as a reference. Suitable bitter substances are preferably aromatic oils, preferably peppermint oil, eucalyptus oil, bitter almond oil, menthol, fruit aroma substances, preferably aroma substances from lemons, oranges, limes, grapefruit or mixtures thereof, and/or denatonium benzoate (Bitrex®); denatonium benzoate is particularly preferably used.

To ensure once daily administration, the dosage form according to the invention comprises the active ingredient at least in part in delayed-release form, wherein the delayed release of the active ingredient may be achieved with the assistance of conventional materials and processes known to the person skilled in the art, for example by embedding the active ingredient in a delayed-release matrix or by applying one or more delayed-release coatings. Active ingredient release must, however, be controlled such that the above-stated conditions are fulfilled in each case, for example that, in the event of correct administration of the dosage form, the active ingredient is virtually completely released before the optionally present component (c) and/or (d) can exert an impairing effect. In particular, release of the active ingredient must ensure analgesic action for at least 24 hours.

If release of the active ingredient from the dosage form according to the invention is controlled with the assistance of at least one delayed-release coating, the delayed-release coating may consist of conventional materials known to the person skilled in the art.

In a preferred embodiment of the dosage forms according to the invention, the delayed-release coating is preferably based on a water-insoluble, optionally modified natural and/or synthetic polymer or on a natural, semi-synthetic or synthetic wax or on a fat or a fatty alcohol or on a mixture of at least two of the above-stated components.

To produce a delayed-release coating, the water-insoluble polymers preferably comprise poly(meth)acrylates, particularly preferably poly(C₁₋₄)-alkyl(meth)acrylates, poly(C₁₋₄)-dialkylamino-(C₁₋₄)-alkyl(meth)acrylates and/or the copolymers thereof, very particularly preferably copolymers of ethyl acrylate and methyl methacrylate with a molar ratio of monomers of 2:1 (Eudragit NE30D®), copolymers of ethyl acrylate, methyl methacrylate and trimethylammonium methyl methacrylate chloride with a molar ratio of monomers of 1:2:0.1 (Eudragit RS®), copolymers of ethyl acrylate, methyl methacrylate and trimethylammonium methyl methacrylate chloride with a molar ratio of monomers of 1:2:0.2 (Eudragit RL®) or a mixture of at least two of these above-stated copolymers. These coating materials are commercially obtainable as 30 wt. % aqueous latex dispersions, i.e. as Eudragit RS30D®, Eudragit NE30D® or Eudragit RL30D® and are preferably also used as such as coating material.

It is likewise preferable to use as water-insoluble polymers for the production of a delayed-release coating for the dosage forms according to the invention polyvinyl acetates optionally in combination with further auxiliary substances. These are commercially obtainable as aqueous dispersions containing 27 wt. % of polyvinyl acetate, 2.5 wt. % of povidone and 0.3 wt. % of sodium lauryl sulfate (Kollicoat SR 30 D®).

In a further preferred embodiment, the delayed-release coatings for the dosage form according to the invention are based on water-insoluble cellulose derivatives, preferably alkylcelluloses such as for example ethylcellulose, or cellulose esters, such as for example cellulose acetate. The coatings of ethylcellulose or cellulose acetate are preferably applied from an aqueous pseudolatex dispersion. Aqueous ethylcellulose pseudolatex dispersions are commercially obtainable as 30 wt. % dispersions (Aquacoate) or as 25 wt. % dispersions (Sureleasee).

If the delayed-release coating is based a water-insoluble, optionally modified natural and/or synthetic polymer, the coating dispersion or solution may comprise, in addition to the corresponding polymer, a conventional physiologically acceptable plasticiser known to the person skilled in the art, in order to reduce the necessary minimum film temperature.

Suitable plasticisers are for example lipophilic diesters from an aliphatic or aromatic dicarboxylic acid with C₆-C₄₀ and an aliphatic alcohol with C₁-C₈, such as for example dibutyl phthalate, diethyl phthalate, dibutyl sebacate or diethyl sebacate, hydrophilic or lipophilic esters of citric acid, such as triethyl citrate, tributyl citrate, acetyl tributyl citrate or acetyl triethyl citrate, polyethylene glycols, propylene glycol, esters of glycerol, such as for example triacetin, Myvacet® (acetylated mono- and diglycerides, C₂₃H₄₄O₅ to C₂₅H₄₇O₇), medium-chain triglycerides (Miglyol®), oleic acid or mixtures of at least two of the stated plasticisers. Aqueous dispersions of Eudragit RS® and optionally Eudragit RL® preferably contain triethyl citrate.

Preferably, a delayed-release coating for the dosage form according to the invention contains plasticisers in quantities of 5 to 50 wt. %, particularly preferably 10 to 40 wt. % and very particularly preferably 10 to 30 wt. %, relative to the quantity of polymer used. In individual cases, for example for cellulose acetate, it is also possible to use larger quantities of plasticisers.

Moreover, a delayed-release coating may comprise further conventional auxiliary substances known to the person skilled in the art, such as for example slip agents, preferably talcum or glycerol monostearate, colouring pigments, preferably iron oxides or titanium dioxide, or surfactants, such as for example Tween 80®.

The release profile obtained for the active ingredient may furthermore be adjusted by conventional options known to the person skilled in the art, such as for example the thickness of the coating or by the use of further auxiliary substances as constituents of the coating. Suitable auxiliary substances are for example hydrophilic or pH-dependent pore formers, such as for example sodium carboxymethylcellulose, cellulose acetate phthalate, hydroxypropylmethylcellulose acetate succinate, lactose, polyethylene glycol or mannitol or water-soluble polymers, such as for example polyvinylpyrrolidone or water-soluble celluloses, preferably hydroxypropylmethylcellulose or hydroxypropylcellulose.

The dosage forms according to the invention for release of the active ingredient may additionally also comprise a coating which is resistant to gastric juices, which dissolves in pH-dependent manner. This coating makes it possible to ensure that the dosage forms according to the invention pass through the stomach undissolved and the active ingredient is not released until it reaches the intestine.

The coating resistant to gastric juices is preferably based on methacrylic acid/alkyl methacrylate copolymers, preferably methyl methacrylate, such as methacrylic acid or ethyl methacrylate copolymers with a molar ratio of the particular monomers of 1:1 to 1:2, such as Eudragit L®, Eudragit S®, Eudragit L30D-55®.

A delayed-release coating may be applied by conventional methods known to the person skilled in the art, such as for example by spraying of solutions, dispersions or suspensions, by melt methods or by powder application methods. The solutions, dispersions or suspensions may be used in the form of aqueous or organic solutions or dispersions. Aqueous dispersions are preferably used in this connection. Organic solvents which may be used are alcohols, for example ethanol or isopropanol, ketones, such as for example acetone, esters, for example ethyl acetate, wherein alcohols and ketones are preferably used. The coating methods are known from the prior art, for example H. Sucker, Georg Thieme Verlag, 1991, pages 347 et seq. They are hereby introduced as a reference and are accordingly deemed to be part of the disclosure.

If the dosage form according to the invention is in multiparticulate form, the delayed-release coating is preferably applied in such a manner that the multiparticulate forms containing the active ingredient are coated, after the production thereof, with the particular polymers and optionally further auxiliary substances from aqueous and/or organic media, preferably from aqueous media, with the assistance of the fluidised bed method and the coating is preferably simultaneously dried at conventional temperatures in the fluidised bed.

A poly(meth)acrylate-based coating is preferably dried at temperatures in the range from 30 to 50° C., particularly preferably from 35 to 45° C. For cellulose-based coatings, such as for example ethylcellulose, drying preferably proceeds at a temperature in the range from 50 to 80° C., particularly preferably in the range from 55 to 65° C. If necessary, drying may additionally be followed by a temperature-controlled treatment in order to obtain a stable release profile.

Delayed release of the active ingredient from the dosage form according to the invention may also be achieved by embedding the active ingredient in a delayed-release matrix.

Materials which may be used for a delayed-release matrix are preferably physiologically acceptable, hydrophilic polymers, preferably cellulose ethers, cellulose esters and/or acrylic resins. Ethylcellulose, methylcellulose, hydroxypropylmethylcellulose, hydroxypropylcellulose, hydroxyethylcellulose, poly(meth)acrylic acid and/or the derivatives thereof, such as the salts, amides or esters thereof, are particularly preferably used.

Where hydrophobic compounds are used as the delayed-release matrix, hydrophobic polymers, waxes, fats, long-chain fatty acids, fatty alcohols or corresponding esters or ethers or mixtures thereof may be used. Mono- or diglycerides of C12-C30 fatty acids and/or C12-C30 fatty alcohols and/or waxes or mixtures thereof are particularly preferably used as hydrophobic compounds.

It is also possible to use mixtures of the above-stated hydrophilic and hydrophobic matrix materials.

Component (b) as a viscosity-increasing agent may preferably also serve as a material for a delayed-release matrix, if this is permitted by the structure of the dosage form according to the invention.

Component (C) and the optionally present component (D), which serve to obtain the breaking strength of at least 500 N, preferably of 1000 N, which is necessary according to the invention, may optionally also serve as additional delayed-release matrix materials.

Corresponding delayed-release compounds and methods for the delayed release of the dosage forms according to the invention and for the application of coatings which are resistant to gastric juices are known to the person skilled in the art, for example from “Coated Pharmaceutical Dosage Forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers. The corresponding literature description is hereby introduced as a reference and is deemed to be part of the disclosure.

The dosage form according to the invention may assume multiparticulate form, preferably the form of microtablets, micropellets, granules, spheroids, beads or pellets, optionally packaged in capsules or press-moulded into tablets. The multiparticulate forms preferably have a size or size distribution in the range from 0.1 to 3 mm, particularly preferably in the range from 0.5 to 2 mm. Depending on the desired dosage form, conventional auxiliary substances (B) are optionally also used for the formulation of the dosage form.

In a further preferred embodiment, the dosage form according to the invention assumes the form of a tablet, a capsule or is in the form of an oral osmotic therapeutic system (OROS), preferably if at least one further abuse-preventing component (a)-(f) is also present.

The abuse-proofed, solid dosage form according to the invention is preferably produced by mixing components (A), (C), optionally (D), optionally at least one of the additional abuse-preventing components (a)-(f) and optionally further auxiliary substances (B), in particular the delayed-release matrix compounds, and, with preceding or simultaneous exposure to heat, forming the resultant mixture, optionally after pelletisation, into the dosage form by application of force.

The pelletisation may be performed by a melt method or by wet pelletisation.

Mixing of components (A), (C) and optionally (D) and of the optionally present components (a)-(f) and optionally the further auxiliary substances (B), in particular the delayed-release matrix compounds may proceed in a mixer known to the person skilled in the art. The mixer may, for example, be a roll mixer, shaking mixer, shear mixer or compulsory mixer.

The resultant mixture is preferably directly formed into the dosage form according to the invention by application of force with preceding or simultaneous exposure to heat.

The mixture may, for example, be formed into tablets by direct tabletting. In direct tabletting with simultaneous exposure to heat, the tabletting tool, i.e. bottom punch, top punch and die, are briefly heated at least to the softening temperature of the polymer (C) and pressed together. In direct tabletting with preceding exposure to heat, the material to be press-moulded is heated immediately prior to tabletting at least to the softening temperature of component (C) and then pressed.

The resultant mixture of components (A), (C), optionally (D), the optionally present components (a)-(f) and optionally further auxiliary substances (B), in particular the delayed-release matrix compounds, may also first be pelletised and then formed into the dosage form according to the invention by application of force with preceding or simultaneous exposure to heat.

It is also possible to form the resultant mixture containing the active ingredient and/or one or more of the pharmaceutically acceptable salts thereof (A) and optionally physiologically acceptable auxiliary substances (B), such as components (a) to (f) and optionally the delayed-release matrix compounds and at least one synthetic or natural polymer (C) and optionally a wax (D), into the dosage form by application of force, optionally to singulate the formed articles and optionally in each case to grade them by size and, after or during heating to at least the softening point of component (C), to expose them to force until the formed articles exhibit a breaking hardness of at least 500 N, preferably of 1000 N, optionally to provide them with a cover, optionally with a delayed-release coating and optionally to mix all the formed articles together again.

If components (c) and/or (d) and/or (f) are present in the dosage form according to the invention, care must be taken to ensure that they are formulated in such a manner or are present in such a low dose that, when correctly administered, the dosage form is able to bring about virtually no effect which impairs the patient or the efficacy of the active ingredient.

If the dosage form according to the invention contains component (d) and/or (f), the dosage must be selected such that, when correctly orally administered, no negative effect is caused. If, however, the intended dosage of the dosage form is exceeded inadvertently, in particular by children, or in the event of abuse, nausea or an inclination to vomit or a bad flavour are produced. The particular quantity of component (d) and/or (f) which can still be tolerated by the patient in the event of correct oral administration may be determined by the person skilled in the art by simple preliminary testing.

If, however, irrespective of the fact that the dosage form according to the invention is virtually impossible to pulverise, the dosage form containing the components (c) and/or (d) and/or (f) is provided with protection, these components should preferably be used at a dosage which is sufficiently high that, when abusively administered, they bring about an intense negative effect on the abuser. This is preferably achieved by spatial separation of at least the active ingredient used from components (c) and/or (d) and/or (f), wherein the active ingredient is preferably present in at least one subunit (X) and components (c) and/or (d) and/or (f) are present in at least one subunit (Y), and wherein, when the dosage form is correctly administered, components (c), (d) and (f) do not exert their effect on taking and/or in the body and the remaining components of the formulation, in particular component (C), are identical.

If the dosage form according to the invention comprises at least 2 of components (c) and (d) or (f), these may each be present in the same or different subunits (Y). Preferably, when present, all the components (c) and (d) and (f) are present in one and the same subunit (Y).

In the case of spatial separation into subunit(s) (X) and subunit(s) (Y) and irrespective of the arrangement of these subunits in the dosage form, a subunit (X) contains the active ingredient in delayed-release form, such that said active ingredient ensures controlled release with once daily administration.

For the purposes of the present invention, subunits are solid formulations, which in each case, apart from conventional auxiliary substances known to the person skilled in the art, contain the active ingredient, at least one polymer (C) and optionally at least one of the optionally present components (a) and/or (b) and/or (e) or in each case at least one polymer (C) and the antagonist(s) and/or emetic(s) and/or component (e) and/or component (f) and optionally at least one of the optionally present components (a) and/or (b) and optionally the delayed-release matrix compounds. Care must here be taken to ensure that each of the subunits is formulated in accordance with the above-stated process.

One substantial advantage of the separated formulation of the active ingredient used from components (c) or (d) or (f) in subunits (X) and (Y) of the dosage form according to the invention is that, when correctly administered, components (c) and/or (d) and/or (f) are hardly released on taking and/or in the body or are released in such small quantities that they exert no effect which impairs the patient or therapeutic success or, on passing through the patient's body, they are only liberated in locations where they cannot be sufficiently absorbed to be effective. When the dosage form is correctly administered, preferably hardly any of components (c) and/or (d) and/or (f) is released into the patient's body or they go unnoticed by the patient.

The person skilled in the art will understand that the above-stated conditions may vary as a function of the particular components (c), (d) and/or (f) used and of the formulation of the subunits or the dosage form. The optimum formulation for the particular dosage form may be determined by simple preliminary testing. What is vital is that each subunit contains the polymer (C) and has been formulated in the stated manner.

Should, contrary to expectations, the abuser succeed in comminuting such a dosage form according to the invention, which comprises components (c) and/or (e) and/or (d) and/or (f) in subunits (Y), for the purpose of abusing the active ingredient and obtain a powder which is to be extracted with a suitable extracting agent, not only the active ingredient but also the particular component (c) and/or (e) and/or (f) and/or (d) will be obtained in a form in which it cannot readily be separated from the active ingredient, such that when the dosage form which has been tampered with is administered, in particular by oral and/or parenteral administration, it will exert its effect immediately on taking and/or in the body combined with an additional negative effect on the abuser corresponding to component (c) and/or (d) and/or (f) or, when the attempt is made to extract the active ingredient, the coloration will act as a deterrent and so prevent abuse of the dosage form.

A dosage form according to the invention, in which the active ingredient is spatially separated from components (c), (d) and/or (e), preferably by formulation in different subunits, may be formulated in many different ways, wherein the corresponding subunits may each be present in the dosage form according to the invention in any desired spatial arrangement relative to one another, provided that the above-stated conditions for the release of components (c) and/or (d), on the one hand, and for release of the active ingredient, namely controlled release for once daily administration, on the other, are fulfilled.

The person skilled in the art will understand that component(s) (a) and/or (b) which are optionally also present may preferably be formulated in the dosage form according to the invention both in the particular subunits (X) and (Y) and in the form of independent subunits (Y′) corresponding to subunits (X) and (Y), provided that neither the abuse-proofing nor the active ingredient release over 24 hours in the event of correct administration is impaired by the nature of the formulation and the polymer (C) is included in the formulation and formulation is carried out in accordance with the above-stated processes.

In a preferred embodiment of the dosage form according to the invention, subunits (X) and (Y) are present in multiparticulate form, wherein microtablets, microcapsules, micropellets, granules, spheroids, beads or pellets are preferred and the same form, i.e. shape, is selected for both subunit (X) and subunit (Y), such that it is not possible to separate subunits (X) from (Y) by mechanical selection. The multiparticulate forms are preferably of a size in the range from 0.1 to 3 mm, preferably of 0.5 to 2 mm.

The subunits (X) and (Y) in multiparticulate form may also preferably be packaged in a capsule or be press-moulded into a tablet, wherein the final formulation in each case proceeds in such a manner that the subunits (X) and (Y) are also retained in the resultant dosage form.

The multiparticulate subunits (X) and (Y) of identical shape should also not be visually distinguishable from one another so that the abuser cannot separate them from one another by simple sorting. This may, for example, be achieved by the application of identical coatings which, apart from this disguising function, may also incorporate further functions, such as, for example, delayed release of the active ingredient or provision of a finish resistant to gastric juices on the particular subunits.

In a further preferred embodiment of the present invention, subunits (X) and (Y) are in each case arranged in layers relative to one another.

The layered subunits (X) and (Y) are preferably arranged for this purpose vertically or horizontally relative to one another in the dosage form according to the invention, wherein in each case one or more layered subunits (X) and one or more layered subunits (Y) may be present in the dosage form, such that, apart from the preferred layer sequences (X)-(Y) or (X)-(Y)-(X), any desired other layer sequences may be considered, optionally in combination with layers containing components (a) and/or (b).

Another preferred dosage form according to the invention is one in which subunit (Y) forms a core which is completely enclosed by the delayed-release subunit (X), wherein a separation layer (Z) may be present between said layers. Such a structure is preferably also suitable for the above-stated multiparticulate forms, wherein both subunits (X) and (Y) and an optionally present separation layer (Z), which must satisfy the hardness requirement according to the invention, are formulated in one and the same multiparticulate form.

In a further preferred embodiment of the dosage form according to the invention, the subunit (X) forms a core, which is enclosed by subunit (Y), wherein the latter comprises at least one channel which leads from the core to the surface of the dosage form.

The dosage form according to the invention may comprise, between one layer of the subunit (X) and one layer of the subunit (Y), in each case one or more, preferably one, optionally swellable separation layer (Z) which serves to separate subunit (X) spatially from (Y).

If the dosage form according to the invention comprises the layered subunits (X) and (Y) and an optionally present separation layer (Z) in an at least partially vertical or horizontal arrangement, the dosage form preferably takes the form of a tablet, a coextrudate or a laminate.

In one particularly preferred embodiment, the entirety of the free surface of subunit (Y) and optionally at least part of the free surface of subunit(s) (X) and optionally at least part of the free surface of the optionally present separation layer(s) (Z) may be coated with at least one barrier layer (Z′) which prevents release of component (c) and/or (e) and/or (d) and/or (f). The barrier layer (Z′) must also fulfil the hardness conditions according to the invention.

Another particularly preferred embodiment of the dosage form according to the invention comprises a vertical or horizontal arrangement of the layers of subunits (X) and (Y) and at least one push layer (p) arranged therebetween, and optionally a separation layer (Z), in which dosage form the entirety of the free surface of the layer structure consisting of subunits (X) and (Y), the push layer and the optionally present separation layer (Z) is provided with a semipermeable coating (E), which is permeable to a release medium, i.e. conventionally a physiological liquid, but substantially impermeable to the active ingredient and to component (c) and/or (d) and/or (f), and wherein this coating (E) comprises at least one opening for release of the active ingredient in the area of subunit (X).

A corresponding dosage form is known to the person skilled in the art, for example under the name oral osmotic therapeutic system (OROS), as are suitable materials and methods for the production thereof, inter alia from U.S. Pat. No. 4,612,008, U.S. Pat. No. 4,765,989 and U.S. Pat. No. 4,783,337. The corresponding description is hereby introduced as a reference and is deemed to be part of the disclosure.

An osmotic dosage form containing an analgesic opioid and a dye as an aversive agent is likewise known to the person skilled in the art from the prior art (WO 03/015531). The tablet core preferably consists of two layers, an opioid-containing layer and a push layer, wherein the push layer contains the dye as the aversive agent. The corresponding description is hereby introduced as a reference and is deemed to be part of the disclosure.

In a further preferred embodiment of the claimed invention, the subunit (X) of the dosage form according to the invention is in the form of a tablet, the edge face and optionally one of the two main faces of which is covered with a barrier layer (Z′) containing component (c) and/or (d) and/or (f).

The person skilled in the art will understand that the auxiliary substances of the subunit(s) (X) or (Y) and of the optionally present separation layer(s) (Z) and/or of the barrier layer(s) (Z′) used in formulating the dosage form according to the invention will vary as a function of the arrangement thereof in the dosage form according to the invention, the mode of administration and as a function of the active ingredient present or of the optionally present components (a) and/or (b) and/or (e) and of component (c) and/or (d) and/or (f), while maintaining release of the active ingredient over 24 hours. The materials which have the requisite properties are in each case known per se to the person skilled in the art.

If release of component (c) and/or (d) and/or (f) from subunit (Y) of the dosage form according to the invention is prevented with the assistance of a cover, preferably a barrier layer, the subunit may consist of conventional materials known to the person skilled in the art, providing that it contains at least one polymer (C) to fulfil the hardness condition of the dosage form according to the invention.

If a corresponding barrier layer (Z′) is not provided to prevent release of component (c) and/or (d) and/or (f), the materials of the subunits should be selected such that release of the particular component (c) and/or (d) from subunit (Y) is virtually ruled out.

The materials which are stated below to be suitable for production of the barrier layer may preferably be used for this purpose. Preferred materials are those which are selected from the group comprising alkylcelluloses, hydroxyalkylcelluloses, glucans, scleroglucans, mannans, xanthans, copolymers of poly[bis(p-carboxyphenoxy)propane and sebacic acid, preferably in a molar ratio of 20:80 (marketed under the name Polifeprosan 20®), carboxymethylcelluloses, cellulose ethers, cellulose esters, nitrocelluloses, polymers based on (meth)acrylic acid and the esters thereof, polyamides, polycarbonates, polyalkylenes, polyalkylene glycols, polyalkylene oxides, polyalkylene terephthalates, polyvinyl alcohols, polyvinyl ethers, polyvinyl esters, halogenated polyvinyls, polyglycolides, polysiloxanes and polyurethanes and the copolymers thereof.

Particularly suitable materials may be selected from the group comprising methylcellulose, ethylcellulose, hydroxypropylcellulose, hydroxypropylmethylcellulose, hydroxybutylmethylcellulose, cellulose acetate, cellulose propionate (of low, medium or high molecular weight), cellulose acetate propionate, cellulose acetate butyrate, cellulose acetate phthalate, carboxymethylcellulose, cellulose triacetate, sodium cellulose sulfate, polymethyl methacrylate, polyethyl methacrylate, polybutyl methacrylate, polyisobutyl methacrylate, polyhexyl methacrylate, polyisodecyl methacrylate, polylauryl methacrylate, polyphenyl methacrylate, polymethyl acrylate, polyisopropyl acrylate, polyisobutyl acrylate, polyoctatdecyl acrylate, polyethylene, low density polyethylene, high density polyethylene, polypropylene, polyethylene glycol, polyethylene oxide, polyethylene terephthalate, polyvinyl alcohol, polyvinyl isobutyl ether, polyvinyl acetate and polyvinyl chloride.

Particularly suitable copolymers may be selected from the group comprising copolymers of butyl methacrylate and isobutyl methacrylate, copolymers of methyl vinyl ether and maleic acid of high molecular weight, copolymers of methyl vinyl ether and maleic acid monoethyl ester, copolymers of methyl vinyl ether and maleic anhydride and copolymers of vinyl alcohol and vinyl acetate.

Further materials which are suitable for formulating the barrier layer are starch-filled polycaprolactone (WO98/20073), aliphatic polyesteramides (DE 19 753 534 A1, DE 19 800 698 A1, EP 0 820 698 A1), aliphatic and aromatic polyester urethanes (DE 19822979), polyhydroxyalkanoates, in particular polyhydroxybutyrates, polyhydroxyvalerates, casein (DE 4 309 528), polylactides and copolylactides (EP 0 980 894 A1). The corresponding descriptions are hereby introduced as a reference and are deemed to be part of the disclosure.

The above-stated materials may optionally be blended with further conventional auxiliary substances known to the person skilled in the art, preferably selected from the group consisting of glyceryl monostearate, semi-synthetic triglyceride derivatives, semi-synthetic glycerides, hydrogenated castor oil, glyceryl palmitostearate, glyceryl behenate, polyvinylpyrrolidone, gelatine, magnesium stearate, stearic acid, sodium stearate, talcum, sodium benzoate, boric acid and colloidal silica, fatty acids, substituted triglycerides, glycerides, polyoxyalkylene glycols and the derivatives thereof.

If the dosage form according to the invention comprises a separation layer (Z′), said layer, like the uncovered subunit (Y), may preferably consist of the above-stated materials described for the barrier layer. The person skilled in the art will understand that release of the active ingredient or of component (c) and/or (d) from the particular subunit may be controlled by the thickness of the separation layer.

Method For Determining Breaking Strength

In order to verify whether a polymer or a wax may be used as component (C) or (D) respectively, the polymer or wax is press-moulded to form a tablet with a diameter of 10 mm and a height of 5 mm using a force of 150 N at a temperature which at least corresponds to the softening point of the polymer or wax and is determined with the assistance of a DSC diagram of the polymer or wax. Using tablets produced in this manner, breaking strength is determined with the apparatus described below in accordance with the method for determining the breaking strength of tablets published in the European Pharmacopoeia 1997, page 143, 144, method no. 2.9.8. The apparatus used for the measurement is a “Zwick Z 2.5” materials tester, Fmax=2.5 kN, draw max. 1150 mm with the setup comprising a column and a spindle, clearance behind of 100 mm, a test speed of 0.1800 mm/min and testControl software. Measurement was performed using a pressure piston with screw-in inserts and a cylinder (diam. 10 mm), a force transducer, (Fmax. 1 kN, diameter=8 mm, class 0.5 from 10 N, class 1 from 2 N to ISO 7500-1, with manufacturers test certificate M to DIN 55350-18, Zwick gross force Fmax=1.45 kN) (all apparatus from Zwick GmbH & Co. KG, Ulm, Germany).

The tablets deemed to be resistant to breaking under a specific load include not only those which have not broken but also those which may have suffered plastic deformation under the action of the force.

The breaking strength of the dosage forms according to the invention is determined using the same measurement method.

The invention is explained below with reference to Examples. These explanations are given merely by way of example and do not restrict the general concept of the invention.

### EXAMPLE 1

### Production of an Abuse-Proofed Tablet Containing (1R,2R)-3-(3-dimethylamino-1-ethyl-2-methyl-propyl)phenol

The quantities of active ingredient hydrochloride, polyethylene oxide powder and hydroxypropylmethylcellulose (Metholose 90 SH 100 000) as the delayed-release matrix material listed in Table 1 were mixed in a free-fall mixer. The tabletting tool, which consisted of die, top punch and bottom punch with a diameter of 13 mm, was heated to 90° C. in a heating cabinet. 600 mg portions of the powder mixture were press-moulded by means of the heated tool, the pressure being maintained for at least 15 seconds.

TABLE 1

| Components | Per tablet | Complete batch |
|---|---|---|
| Active ingredient HCl | 200.0 mg | 60.0 g |
| Polyethylene oxide, NF, MW 7 000 000 | 360.0 mg | 138.0 g |
| (Polyox WSR 303, Dow Chemicals) | | |
| Hydroxypropylmethylcellulose 100 000 | 40.0 mg | 12.0 g |
| mPas (Metholose 90 SH 100 000) | | |
| Total weight | 600.0 mg | 210.0 g |

The breaking strength of the tablets was determined using the above-described method. No breakage occurred when a force of 500 N was applied. The tablets could not be comminuted using a hammer, nor with the assistance of a pestle and mortar.

In vitro release from the tablets produced

In vitro release of active ingredient hydrochloride from the tablets produced was determined in a paddle stirrer apparatus with sinker according to the method described in the European Pharmacopoeia. The temperature of the release medium was 37° C. and the rotational speed of the stirrer 75 min⁻¹. The release medium used was intestinal juice, pH 6.8. The quantity of active ingredient hydrochloride released in each case into the dissolution medium at any one time was determined by spectrophotometry. The percentage released quantity, relative to the total quantity of active material hydrochloride, at each point in time is shown in Table 2.

TABLE 2

| Time, minutes | Released quantity, wt. % |
|---|---|
| 30 | 12 |
| 240 | 42 |
| 480 | 65 |
| 720 | 80 |
| 1080 | 94 |
| 1440 | 99 |

1. An abuse-proofed, oral dosage form with controlled release of (1R,2R)-3-(3-dimethylamino-1-ethyl-2-methyl-propyl)phenol for once daily administration, comprising (1R,2R)-3-(3-dimethylamino-1-ethyl-2-methyl-propyl)phenol and/or at least one of the pharmaceutically acceptable salts or derivatives thereof (A), at least one synthetic or natural polymer (C), optionally delayed-release matrix auxiliary substances, optionally physiologically acceptable auxiliary substances (B), optionally a wax (D) and optionally at least one delayed-release coating, component (C) or (D) in each case exhibiting a breaking strength of at least 500 N.

2. A dosage form according to claim 1, which is in the form of a tablet.

3. A dosage form according to claim 1, which is in multiparticulate form, optionally press-molded into tablets or packaged in capsules.

4. A dosage form according to claim 1, wherein the polymer (C) is at least one polymer selected from among the group consisting of polyethylene oxides, polyethylenes, polypropylenes, polyvinyl chlorides, polycarbonates, polystyrenes, polyacrylates and the copolymers thereof.

5. A dosage form according to claim 4, wherein the polyethylene oxide is of high molecular weight.

6. A dosage form according to claim 4, wherein the polymer (C) is a water-soluble or water-swellable polymer.

7. A dosage form according to claim 1, wherein the wax (D) is at least one natural, semi-synthetic or synthetic wax with a softening point of 60° C.

8. A dosage form according to claim 7, wherein the wax (D) is carnauba wax or beeswax.

9. A dosage form according to claim 1, wherein the component(s) (C) and optionally (D) are present in such quantities that the dosage form exhibits a breaking strength of at least 500 N.

10. A dosage form according to claim 1, wherein the active ingredient is present in a delayed-release matrix.

11. A dosage form according to claim 10, wherein component (C) and/or component (D) also serves as a material for the delayed-release matrix component.

12. A dosage form according to claim 1, wherein at least one auxiliary substance (B) serves as a material for the delayed-release matrix.

13. A dosage form according to claim 1, which comprises a delayed-release coating.

14. A dosage form according to claim 1, which comprises at least one of the following components (a)-(f) as the auxiliary substance (B):
(a) at least one substance which irritates the nasal passages and/or pharynx,
(b) at least one viscosity-increasing agent, which, with the assistance of a necessary minimum quantity of an aqueous liquid forms a gel which remains visually distinguishable when introduced into a further quantity of an aqueous liquid,
(c) at least one antagonist for each of the opioids or opiates with potential for abuse,
(d) at least one emetic,
(e) at least one dye as an aversive agent, and
(f) at least one bitter substance.

15. A dosage form according to claim 14, wherein the viscosity-increasing agent is at least one polymer selected from among the group consisting of carboxymethylcellulose sodium, polyacrylic acid, locust bean flour, pectin, waxy maize starch, alginate, guar flour, iota-carrageenan, karaya gum, gellan gum, galactomannan, tara stone flour, propylene glycol alginate, hyaluronate, tragacanth, tara gum, fermented polysaccharide welan gum and xanthan.

16. A dosage form according to claim 15, wherein component (C) serves as an additional viscosity-increasing agent.

17. A process for the production of a dosage form according to claim 1, comprising
(1) mixing components (A), (C), optionally (B) and optionally (D) and optionally delayed-release matrix compounds and
(2) forming the resultant mixture, optionally after pelletisation, into the dosage form by application of force and with preceding or simultaneous exposure to heat and optionally providing the dosage form with a delayed-release coating.

18. A process according to claim 17, wherein pelletisation is performed by a melt method.

19. A process according to claim 17, wherein pelletisation is performed by wet pelletisation.

20. A process for the production of a dosage form according to claim 1, comprising
(1) forming a mixture containing components (A), (C), optionally (B) and optionally (D) and optionally delayed-release matrix compounds into formed articles by application of force,
(2) optionally singulating the formed articles obtained and optionally in each case grading by size and
(3) after or during heating to at least the softening point of component (C), exposing the formed articles to force until the formed articles exhibit breaking hardness of at least 500 N, and
(4) optionally providing with a cover, optionally a delayed-release coating and optionally mixing the formed articles all together again.

21. A dosage form obtainable by a process according to claim 17.

22. A dosage form obtainable by a process according to claim 20.
